(19) United States
(12) Patent Application Publication
(10) Pub. No.: US 2023/0052222 A1
(43) Pub. Date: Feb. 16, 2023

(54) HEALTH CARE INFORMATION NETWORK OF NETWORKS

(71) Applicants: Cora ALISUAG, Washington, DC (US); CORAnet Solutions, Inc., Washingto, DC (US)

(72) Inventor: Cora ALISUAG, Washington, DC (US)

(21) Appl. No.: 17/796,576

(22) PCT Filed: Jan. 29, 2021
(86) PCT No.: PCT/US2021/015934

§ 371 Date: Jul. 29, 2022

Related U.S. Application Data
(60) Provisional application No. 62/967,448, filed on Jan. 29, 2020.
(60) Provisional application No. 62/969,564, filed on Feb. 3, 2020.

(51) Int. Cl. G16H 10/60; G06F 16/27; H04L 67/12

(52) U.S. Cl. CPC G16H 10/60; G06F 16/27; H04L 67/12

(57) ABSTRACT

A network of networks enables seamless and effective aggregation of a complete electronic health record on a user's personal device and sharing of the complete medical history among health organizations as authorized by the user. A complete health record includes all diagnoses, testing, and treatment related to routine medical services, ambulatory medical services, emergency medical services, telemedicine, data collected from personal monitors, and/or data entered by user. Also provided is automatic updating of the electronic health record to include information from new medical services.

### FIELD OF THE INVENTION

This disclosure relates generally to a mobile personal health information exchange. More specifically, the mobile personal health information exchange provides an electronic health record aggregated from multiple sources of health records, to which access is controlled by the user, or patient, and which is updated automatically.

### BACKGROUND

Facing challenges in quality of care and runaway costs, resulting in unsustainable fiscal challenges, healthcare systems are under a financial burden. Without mitigation, the continued growth of this burden ultimately leads to higher costs or lower quality care to the patient. Lessening this burden, while improving quality of care, in many instances, may be as simple as collecting a complete medical history from or for an individual. When an individual sees a healthcare provider under normal circumstance, such as for a scheduled appointment to treat an illness, a complete medical history is collected before rendering care. The value of obtaining a complete medical history, and the cost savings it provides, cannot be understated. More valuable in diagnosis than laboratory tests, seventy-six percent (76%) of diagnoses result from medical histories compared to eleven percent (11%) from laboratory test and twelve percent (12%) from physical examination. (Peterson et al., “Contributions of the history, physical examination, and laboratory investigation in making medical diagnoses,” Western Journal of Medicine, 1992 February; 156(2): 163-165.) Filling out forms may seem annoying and time consuming, but it allows healthcare providers to make accurate diagnoses without relying on costly tests and examinations.

A complete medical history would ideally include all diagnoses, testing, and treatment records from all sources of medical treatment, including ambulatory medical services, emergency medical services, and routine doctor visits for check-ups or minor illnesses. It would also be desirable to include radiological and other images, results from any laboratory testing, and video and audio from telemedicine services. A growing market in medical patient monitors allows a patient to monitor a variety of real-time biometric and/or medical data such as blood pressure, heart rate, electrophysiology, body temperature, blood oxygen saturation, posture, physical activities, and various sweating biomarkers. The ability to process and add such real-time data to an electronic medical history would further improve the usefulness of a complete medical record. Ideally, processing of such real-time data would include a comparison to standardized medical data including expected values of measured characteristics for the patient's demographic group and identification of any possible health-related abnormalities.

In addition to having a complete medical history for a patient available, it would further be desirable for such medical history to be updated automatically with no affirmative action required by the patient. “As widespread adoption of the electronic health record (EHR) takes place in US medical practice, many health care experts have emphasized the promising capabilities of the EHR to foster patient activation, which is a characteristic of patients who view themselves as active collaborators in their own health care management . . . . After reviewing their visit notes, patients reported feeling more in control of their care.” (White, Amina, and Marion Danis. “Enhancing patient-centered communication and collaboration by using the electronic health record in the examination room.” JAMA vol. 309, 22 (2013): 2327-8. doi: 10.1001/jama.2013.6030.) Automated availability of complete, up-to-date health records not only reduces cost and effort for all involved in the health care process but promotes better long-term health through the patient's review and control of these records.

### BRIEF DESCRIPTION OF THE DRAWINGS

The present disclosure is illustrated by way of example, and not by way of limitation, in the accompanying drawings, in which like reference characters refer to like parts throughout, and in which:

FIG. 1 shows an architecture of system comprising a network of networks. Descriptions of FIG. 2-FIG. 5 in the DETAILED DESCRIPTION refer to reference numbers in FIG. 1 to provide context for the flow of electronic health record information in each process represented.

FIG. 2 is a flow diagram for updating electronic health records in a personal health information exchange system applied in a personal repository.

FIG. 3 is a flow diagram for updating electronic health records in a personal health information exchange system related to delivery of medical services.

FIG. 4 is a flow diagram for updating electronic health records in a personal health information exchange system related to emergency medical services.

FIG. 5 is a flow diagram for updating electronic health records in a personal health information exchange system related to use of patient monitors.

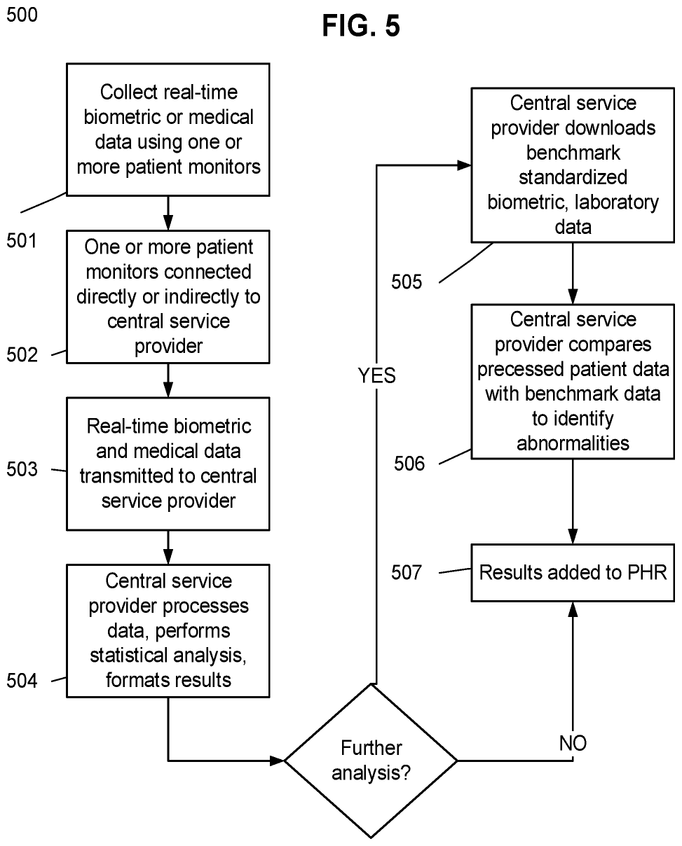

### DETAILED DESCRIPTION

Various aspects will now be described with reference to specific forms selected for purposes of illustration. It will be appreciated that the spirit and scope of the architectures, systems and methods disclosed herein are not limited to the selected forms.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

Each of the following terms written in singular grammatical form: “a,” “an,” and “the,” as used herein, may also refer to, and encompass, a plurality of the stated entity or object, unless otherwise specifically defined or stated herein, or, unless the context clearly dictates otherwise.

### Types of Records

“Electronic medical record” (EMR) typically refers to a digital version of a patient's charts created by individual health facilities or providers. Unlike paper charts, EMRs can help clinicians track and monitor data over time and identify patients with particular needs. The biggest drawbacks to EMRs is that they are kept by individual providers of medical services and do not typically support interoperability or the exchange of information with other providers. Although a digitally stored EMR is an improvement over traditional paper charts, its usefulness is limited by a lack of standards-based interoperable data. An EMR is generally a patient's record associated with a specific provider.

“Electronic health record” (EHR), like an EMR, is a digital version of a patient's chart. An EHR, however, differs from an EMR in that it includes data from all medical service providers and facilities involved in a patient's care and participating in the relevant EHR system. All authorized providers and staff can access information in an EHR to treat a patient. An EHR can also include information about: diagnoses, allergies, medications, treatment plans, immunization dates, test results (including radiology and other images). Ideally, an EHR is complete medical history associated with a patient and is not specific to a specific provider. For example, a patient receiving care for a back condition might receive care from a primary care physician, an orthopedic specialist, a magnetic resonance imaging (MRI) facility, and a physical therapist. Each of these providers would contribute information about the patient's treatment to the same EHR. This makes the EHR essential for data sharing and coordination of care. As widespread adoption of the EHR takes place in U.S. medical practice, many health care experts have emphasized the promising capabilities of the EHR to foster active participation of the patient in managing their own health care.

“Personal health record” (PHR), like an EMR or EHR is likely maintained digitally but unlike the EMR or EHR is controlled by the patient instead of the medical service provider(s). Data kept within a PHR may come from an EHR, but the patient is in charge of management of and access to the PHR. A patient can enter information into their PHR themselves, such as their lifestyle habits, over-the-counter medications, home monitoring devices, wearables, biometrics devices, and additional care providers that may not be part of an EHR system.

As used herein, “PHR” will refer to the patient's EHR stored on the patient's personal repository, and EHR refers to the patient's health history stored in the repository of one or more medical service providers. One aspect of the invention disclosed herein is that although the patient controls the PHR, the patient authorizes synchronization of the PHR and EHR within the system such that the PHR is automatically and periodically updated to reflect a complete medical history and the EHRs of authorized providers will contain the same updated information.

### Sources of Medical Data

“Ambulatory medical services,” as used herein, means medical services performed on an outpatient basis, without admission to a hospital or other facility. Ambulatory patient services include, but are not limited to, visits to specialists, physical therapy, trips to emergency room when the patient is mobile, diagnostic tests, treatments, or rehabilitation visits. The location at which ambulatory services can be delivered includes doctor's office, clinic, emergency room, and outpatient hospital department. Nonlimiting examples of ambulatory care include blood tests, biopsy, chemotherapy, colonoscopy, CT scan, mammograms, minor surgical procedures, radiation treatments, ultrasound imaging, and x-rays.

“Emergency medical services,” as used herein, means provision of medical services to the patient when the patient is suffering from a medical condition manifesting itself by acute symptoms of severity (including severe pain), such that a prudent layperson, who possesses an average knowledge of health and medicine, could reasonably expect the absence of immediate medical attention to result in placing the health of the individual in serious jeopardy. Such medical conditions requiring emergency services include natural events, such as a heart attack, diabetic shock, or traumatic events, such as an automobile accident, stabbing, or gunshot wound. In either case, an aspect of emergency services provided in the context of the personal health information exchange system is that the event precipitating the need for emergency services occurs at a location distant from medical service providers, such as when the patient is at home, at work, or even on vacation distant from home. Additionally, the patient requiring emergency services may be unconscious or otherwise incapacitated, such that the patient is unable to assist in their own care.

“Telemedicine services,” as used herein, means the remote diagnosis and treatment of patients by means of telecommunications technology. Telemedicine is the exchange of medical information from one location to another using electronic communication in order to improve the health status of a patient. Such electronic communication includes wireless tools, email, two-way video, smartphones, and other methods of telecommunications technology. As early as the 1980's, hospitals began extending their services to patients in remote locations. Since then, telemedicine has become an integrated part of specialty departments, hospitals, private doctor offices, and home health care. A patient can receive the benefits of telemedicine at home, at work, or even when traveling. The products and services of telemedicine are not a separate medical specialty but are instead part of the expanded platform of healthcare settings providing an alternative means for delivering healthcare. Billing and coding for remote medical services provided through telemedicine are typically the same or very similar to and those for equivalent services delivered on site.

“Routine medical services,” as used herein, means traditional delivery of medical services for check-ups and/or diagnosis, testing, and treatment for minor illnesses (e.g. a normal visit to a primary care physician). Routine medical services can also include prescribing and filling prescriptions for medication either by a physician or pharmacist.

“Standardized medical data,” as used herein, means a database maintained by a provider that contains the compiled biometric and medical data of a statistically significant number of people in a standardized format. The database can contain biometric and/or medical data such as, but not limited to, one or more of blood pressure, heart rate, electrophysiology, body temperature, blood oxygen saturation, posture, physical activities, and various sweating biomarkers. The data is categorized by one or more of height, weight, age, demographic data, geographical region, or other relevant factors and statistically processed to provide benchmark information for comparison to real-time biometric and/or medical data gathered from the patient in order to identify any possible health-related abnormalities.

### Components of the Personal Health Information Exchange

“Application Programming Interface” or “API,” as used herein, is a software-to-software interface and not a user interface with software. An API is a set of commands, functions, protocols, and objects that can be used to interact with an external system. APIs are used to control access to resources such as hardware devices and software functions that an application may not necessarily have permission to use, which is why APIs often play a big role in security. APIs are also used for communication between services. With APIs, applications communicate without any user knowledge or intervention. The user sees only one interface, but behind the scenes, many applications may be working together using APIs. This type of integration is often called “seamless,” since the user never notices when software functions are handed from one application to another. An API resembles Software as a Service (SaaS), since software developers do not have to start from scratch every time they write a program. Instead of building one core application that tries to do everything, the application can contract out certain responsibilities to remote software that already performs specific tasks efficiently. An API can be configured to maintaining a ledger associated with an EHR. In addition to maintaining the ledger, the API can be configured to update EHRs throughout all or a portion of the network of networks. For instance, when a participant makes a change to a medical history the API can send instructions directing medical histories stored in all or a portion of the network of networks be updated to reflect the change.

“Cartridge,” as used herein, means software components that extend a server. Cartridge constituents reside on the server or are accessed from the server. Packaging domain-specific component expertise in a data cartridge allows the cartridge to access the corporate information repository and add both organizational and operational value to the data. Such software components are applications that can be “plugged” into other software components, and which are themselves “pluggable.” Data cartridges are server-based, extend the server, are integrated with the server, are packaged to address access issues of various target users.

“CCD,” as used herein, means Continuity of Care Document, which is based on the HL7 CDA architecture. The CCD is a joint effort of HL7 International and ASTM. CCD fosters interoperability of clinical data by allowing physicians to send electronic medical information to other providers without loss of meaning and enabling improvement of patient care. CCD is an implementation guide for sharing Continuity of Care Record (CCR) patient summary data using the HL7 Version 3 Clinical Document Architecture (CDA), Release 2. CCD establishes a rich set of templates representing the typical sections of a summary record and expresses these templates as constraints on CDA. These same templates for vital signs, family history, plan of care, and so on, can then be reused in other CDA document types, establishing interoperability across a wide range of clinical use cases. Some of its benefits are: allowing physicians to send electronic medical information to other providers without loss of meaning and provides a “snapshot in time,” constraining a summary of the pertinent clinical, demographic, and administrative data for a specific patient.

“CDA,” as used herein, means Clinical Document Architecture, which is a document standard governed by the HL7 organization. The HL7 CDA is a document markup standard that specifies the structure and semantics of “clinical documents” for the purpose of exchange. It supports exchange of human-readable documents between users, including those with different levels of technical sophistication.

“Central service provider,” as used herein, is a connection point for multiple networks and can further host multiple APIs and/or cartridges to facilitate communication between the networks and perform processing on data exchanged between networks. A preferred central service provider is configured to enable authenticated access to one or more medical EHR systems. For instance, a central service provided can be configured to store profiles for various networks, healthcare providers, and/or individuals and to verify ownership of a profile using one more of personal identification numbers, passwords, biometrics and/or unique numbers. Once a network is verified, a central service provider can be configured to provide an approval code enabling establishment of a communication link between the requesting and transmitting networks, enabling transmission of the medical history. In some embodiments, the central service provider does not require purchase of an information technology (IT) product by a user or organization, but instead builds, operates, and manages IT products, which are bundled and delivered as a service/solution. A customer can access this type of service/solution from a central service provider via several different sourcing models, including, but not limited to, a monthly or annual subscription fee.

“Communication link,” as used herein, means any form of electronic connection suitable for the secure transmission of data between a health organization network and a central service provider. Communication links are not particularly limited and can include any communication channel through which data can be transmitted. For instance, communication links can comprise a direct connection through telephone lines, satellite links, radio transmissions digital subscriber line, internet, cellular networks, cloud-based communications and/or other transmission channels, or an indirect connection which can be any combination of the foregoing. Examples of security protocols for these links include, but are not limited to, Secure Electronic Transactions (SET), Secure Socket Layer (SSL), Secure Hypertext Transfer Protocol (S-HTTP), Secure Shell (SSH-2), and IP Security (IPSec). Communication links can comprise Transport Layer Security (TLS 1.2 &TLS 1.3) protocol for encrypted transmissions of EHR data. A preferred communication link protocol can be two-way TLS, also known as TLS with client certificate authentication. A preferred configuration would utilize 2048-bit SSL certificates along with −256-bit encryption.

“Emergency Health Information Exchange” network or “EHIE” means a health organization network, which is part of the personal health information exchange network used by health organizations for the purpose of exchanging personal health information needed in critical, emergency situations and which can be accessed without physical action by the patient. All or a portion of the patient's EHR is maintained on the EHIE. In any event, the portion of the EHR available through the EHIE is at least enough for first responders to provide standard care required prior to transport of the patient to an emergency room at a hospital or other similar facility. Despite the convenience and access to data personal repositories provide, often in emergency and/or disaster situations, a user may be unable to access a personal repository. For instance, a user may be unconscious and/or without cellular service. In such instances, healthcare providers may need to access at least a portion of the user's EHR, such as lifesaving information only (name, date of birth, gender, blood type, allergies, and current medications. Through communication link associated with the EHIE, first responders and/or emergency room personnel can obtain a victim's EHR as pre-authorized by the user. To facilitate obtaining information from an unresponsive user and/or a user without that ability to access their personal repository, the EHIE can comprise a communication link to an identification repository. For instance, the identification repository can comprise a National Master Patient Index and/or driver registry. The link can be configured to enable the transmissions of user identifying information such as, but not limited to, a photograph of victim, fingerprint, biometric data, and/or other data uniquely identifying an individual. Once identified, all or a portion of the user's EHR, as pre-authorized by the user, can be obtained through the network of networks.

“Health organization network,” as used herein, means a linkage of files or servers within one organization or a linkage of multiple organizations using a common platform for storing and accessing EHRs. Transmission of EHRs between health organization networks requires files in CCD format or additional APIs residing on the central service provider linking the networks to facilitate translation. Health organization networks can comprise a network of multiple clinics, offices, hospitals, and/or other facilities managing EHRs. A health organization network can also be an individual provider of ambulatory medical services, emergency medical services, telemedicine services, or routine medical services. For instance, one or more of health organization networks can comprise a clinic housing the medical histories of its patients on a single local server. It also possible one or more of health organization networks can comprise cloud storage holding medical histories. Regardless of complexity, size and manner in which EHRs are stored, each health organization network contains one or more repositories of EHRs. By communicating via communication links through central service provider, health organization networks form a network of networks enabling the exchange of medical histories. Regardless of communication channels employed, health organization networks comprise repositories of EHRs, and during the course of providing medical care, these EHRs are updated. The EHIE is a specific type of health organization network.

“HL7,” as used herein, means a standard for exchanging information between medical applications. It defines a format for the transmission of health-related information. HL7 is an international standard used to transfer and share data between various healthcare providers and helps bridge the gap between health information technology applications by allowing easier and more efficient sharing of healthcare data. HL7 Fast Healthcare Interoperability Resources (FHIR™) refers to an interoperability standard facilitating the exchange of healthcare information between healthcare providers, patients, caregivers, payers, researchers, and any other party involved in the healthcare system. HL7 FHIR™ represents granular clinical concepts which can be used to exchange to quickly and effectively solve problems in healthcare and related process and includes the basic elements of healthcare, like patients, diagnostic reports, admissions, medications, and problem lists, along with their typical participants. HL7 FHIR™ can also support a range of more complex clinical models. Resources are generally based on thorough requirements gathering, formal analysis, and extensive cross-mapping to other relevant standards.

“Ledger,” as used herein, means a transaction record related to an EHR. Distributed ledger technology is the use of multiple ledgers that are synchronized to provide synchronized EHRs and PHR. A ledger comprises a system of codes detailing one or more of the origin of a transfer of an EHR, the destination of the transfer of an EHR, changes made to an EHR, the origin of change to an EHR, and/or route of transmission. The codes can comprise institution code, a state and/or country code, a location code, and/or a branch code. Regardless of the communication link, each network within a peer-to-peer network will maintain ledgers reflecting changes to the medical histories. Similar to the common use of the word ledger, a ledger in the context of Distributed Ledger Technology is simply an electronic record or collection of transactions. An important distinction is that the ledger is not a collection of assets. While assets are a part of transactions, the ledger records the transactions related to those assets.

“Master patient index,” as used herein, means a form of customer data integration (CDI) specific to the healthcare industry. Healthcare organizations and groups use Enterprise Master Patient Index (EMPI) to identify, match, merge, de-duplicate, and cleanse patient records to create a master index that may be used to obtain a complete and single view of a patient.

“Patient,” “user,” and “victim” are used interchangeably herein.

“Patient monitors,” as used herein, means devices, often using wearable technology, suited for monitoring a variety of a patient's real-time biometric and/or medical data such as, but not limited to, blood pressure, heart rate, electrophysiology, body temperature, blood oxygen saturation, posture, physical activities, stress level, EKG patterns, weight tracking, and various sweating biomarkers. Data from such patient monitors can be collected for analysis by storage on the patient monitor for later download, transmitted to the patient's mobile device, such as but not limited to a smartphone, or transmitted directly to a service associated with a particular patient monitor. In any case, there will be software, associated with the device, tailored for processing the specific type of biometric and/or medical data being collected. In conjunction with the collection of real-time biometric and/or medical data from the patient, standardized medical data can be accessed for comparison of the patient's real-time biometric and/or medical data to the expected real-time biometric and/or medical data for the patient to identify any possible health-related abnormalities.

“Personal repository,” as used herein, means any device having a storage medium and user interface permitting the medical history to be display. Accordingly, personal repositories can comprise smartphones, cell phones, laptops, desktop computers and/or patient monitors. Regardless of the specific form, personal repositories enable an individual to share their PHR or EHR with a healthcare provider.

“Providers of medical services,” as used herein, means entities engaged in providing ambulatory medical services, emergency medical services, telemedicine services, or routine medical services, and that also maintain EHRs for customers/patients.

“Server enhancement,” as used herein, means one or more APIs, one or more cartridges, or combinations thereof.

### Description of System Embodiment

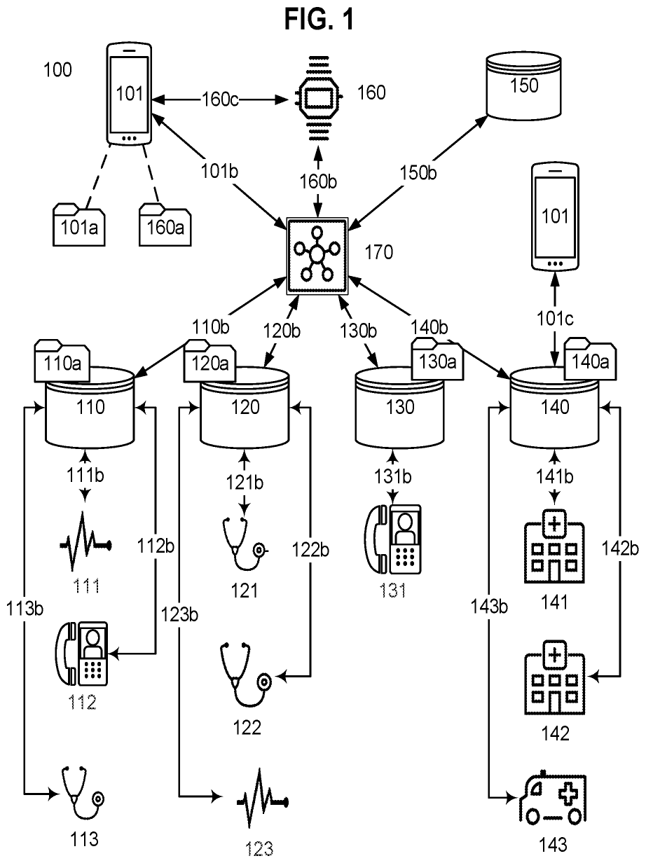

Providing seamless sharing of medical histories, the architecture comprises a central service provider 170, as shown in FIG. 1. Configured to operate as gateway, central service provider 170 establishes communication links 110b, 120b, and 130b between different health organization networks 110, 120, and 130, respectively.

In addition to health organization networks 110, 120, and 130, the architecture can comprise a network of patient monitors 160 communicating with one or more of the health organization networks 110, 120, and 130 via central service provider 170 through one or more communication links 110b, 120b, and 130, respectively. All or a portion of the patient monitors 160 can utilize a computer, such as a cell phone or laptop, to communicate with central service provider 101. Regardless of whether patient monitors communicate with service provider 101 directly or indirectly, patient monitors 160 collect biometric and/or other medical data. Transmitting the collected data through service provider 170 to all or a portion of health organization networks 110, 120, and 130, patient monitors 104 update a medical history.

The network of networks further comprises one or more personal repositories 101 storing medical histories. Through communication link 102, personal repositories 101 can obtain and store one or more medical histories.

For when a user is unresponsive and/or lacks the ability to access the personal repository, the network of networks can further comprise an EHIE network 140. Through communication link 140b, emergency medical services providers can obtain all or a portion of the user's medical history. Additionally, the EHIE network 140 can comprise a link to an identification repository to aid in identifying the incapacitated user.

Accuracy of the medical records obtained through the network of networks is essential. Maintaining ledgers of transactions on one of more networks 110, 120, 130, and/or 140 can facilitate such accuracy. Accordingly, the architecture and/or system can comprise one or more ledgers stored within one or more of networks 110, 120, 130, and/or 140. As to facilitate maintaining the ledgers in a trusted state, all or a portion of networks 110, 120, 130, and/or 140 can be organized as peer-to-peer networks and/or make use of cartridges and/or distributed ledger technology. Regardless of the organization of networks 110, 120, 130, and/or 140, central service provider 170 is configured to provide a consistent server enhancement for each network of the network of networks. Each network, ideally, but not necessarily, utilizes the same server enhancement to interface with central service provider 170. The single and/or multiple server enhancements provided by central repository 170 can be configured to provide a private subnetwork between networks through all or a portion of communication links 170.

Providing a communication link between networks, the central repository can be configured to only enable passing of information. Merely passing information between networks of the network of networks, central service provider 170 can be configured not to store medical histories after transmission to the receiving network. Accordingly, central service provider 170 can be configured to only transiently store information identifying a patient, document, and/or medical history necessary for entry into the ledger. Once the receiving and/or transmitting networks have verified successful updating of the ledgers, central service provider 170 can be configured to delete all or a portion of the transmitted medical history and/or accompanying ledger information.

### Description of System Method

In an embodiment shown in FIG. 1, the focal point of the personal health information exchange (PHIE) system 100 is the personal repository 101, which is an electronic mobile device, such as, but not limited to, a smartphone. The PHIE system 100 functions to aggregate and transmit to the personal repository 101 a comprehensive and current EHR, which is the context of the patient's personal repository is equivalent to and interchangeable with PHR 101a. The PHIE system 100 functions to automatically update the PHR 101a based on an update to the patient's EHR maintained by one or more health organization networks 110, 120, 130, and/or 140, which subscribes to the PHIE system 100.

The user, or patient, is in control of the personal repository 101. The personal repository 101 is connected to a central service provider 170 through a communication link 101b related to the personal repository 101. The central service provider 170, in turn, connects to one or more health organization networks: health organization network 110, which maintains a copy of patient's EHR 110a, through a communication link 110b related to health organization network 110; health organization network 120, which maintains a copy of patient's EHR 120a, through a communication link 120b related to health organization network 120; health organization network 130, which maintains a copy of patient's EHR 130a, through a communication link 130b related to health organization network 130; and emergency health information exchange network 140, which maintains a copy of all or a portion of a patient's EHR 140a, through a communication link 140b related to health organization network 140.

In the embodiment of the PHIE 100 shown in FIG. 1, health organization network 110, in turn, is connected to one or more providers of medical services: an ambulatory services provider 111, which maintains a copy of patient's EHR 111a, through a communication link 111b related to the ambulatory services provider 111; a telemedicine services provider 112, which maintains a copy of patient's EHR 112a, through a communication link 112b related to the telemedicine services provider 112; and/or a routine medical services provider 113, which maintains a copy of patient's EHR 113a, through a communication link 113b related to the routine medical services provider 113.

In the embodiment of the PHIE 100 shown in FIG. 1, health organization network 120, in turn, is connected to one or more providers of medical services: a routine medical services provider 121, which maintains a copy of patient's EHR 121a, through a communication link 121b related to the routine medical services provider 121; a routine medical services provider 122, which maintains a copy of patient's EHR 122a, through a communication link 122b related to the routine medical services provider 122; and a telemedicine services provider 121, which maintains a copy of patient's EHR 121a, through a communication link 121b related to the telemedicine services provider 121.

In the embodiment of the PHIE 100 shown in FIG. 1, health organization network 130, in turn, is connected to a telemedicine services provider 131, which maintains a copy of patient's EHR 131a, through a communication link 131b related to the routine medical services provider 131. In this instance, telemedicine services provider 131 might not be part of a broader health organization network with other members. Health organization network 130, in this case could be a network owned and operated by telemedicine services provider 131, such that telemedicine services provider 131 is linked directly to the central services provider 170.

In the embodiment of the PHIE 100 shown in FIG. 1, health organization network 140, in turn, is connected to one or more providers of emergency medical services: a hospital emergency room 141, which maintains a copy of patient's EHR 141a, through a communication link 141b related to the hospital emergency room 141; a hospital emergency room 142, which maintains a copy of patient's EHR 142a, through a communication link 142b related to the hospital emergency room 142; and a first responder services provider 143, which maintains a copy of patient's EHR 143a, through a communication link 143b related to the first responder services provider 143. If the patient is conscious and the patient personal repository 101 is available, providers of emergency medical services 141, 142, 143 can access critical patient information as previously authorized by patient (i.e. when subscribing to the PHIE) through communication link 101c.

The above description are nonlimiting illustrations of the possible variations of medical services included in the PHIE disclosed herein. An individual health organization network can be a single provider of ambulatory, telemedicine, routine, or emergency services. Alternatively, an individual health organization network can comprise one or more providers of a single service type selected from ambulatory, telemedicine, routine, or emergency services. In other cases, an individual health organization network can comprise one or more providers, in any combination, selected from one or more of ambulatory, telemedicine, routine, and/or emergency services.

In some embodiments, each medical services provider is part of a different health organization network than each of the other medical services providers, in which case one or more APIs residing on the central service provider 170 will enable the connection between each medical services provider and the central service provider 170 by way of the communication link related to each such medical services provider to form a network of networks. The one or more APIs hosted by the central service provider 170 can be configured to provide a private subnetwork between networks through all or a portion of communication links 110b, 120b, 130b, and/or -140b.

EHR and PHR information and updates are securely transmitted through the network of networks. Accuracy of the EHRs 110a, 120a, 130a, and/or 140a maintained by each health organization network 110, 120, 130, and/or 140 is assured by one or more ledgers of transactions related to each EHR and PHR. The PHIE system 100 comprises one or more ledgers stored within one or more of networks of each provider of medical services 110, 120, 130, and/or 140. All or a portion of providers of medical services 110, 120, 130, and/or 140 can be organized as peer-to-peer networks in order to maintain the ledgers in a trusted state. The central service provider 170 is configured to provide a consistent server enhancement for each provider of medical services 110, 120, 130, and/or 140 joined in the network of networks. The one or more APIs residing on the central service provider 170 can further be configured to provide a private subnetwork between networks through all or a portion of communication links 110b, 120b, 130b, and/or 140b.

In an embodiment, the central service provider 170 is configured to transmit information. Acting as a gateway between networks of the providers of medical services 110, 120, 130, and/or 140, central service provider 170 is configured to store EHR information transiently after transmission to the receiving service provider and erased once the receiving and/or transmitting service providers have verified successful updating of the relevant ledgers of transactions.

Each provider of medical services 110, 120, 130, and/or 140 within a peer-to-peer network will maintain ledgers reflecting changes to the EHRs 110a, 120a, 130a, and/or 140a. Besides maintaining the ledger, the one or more server enhancements are configured to update EHRs 110a, 120a, 130a, and/or 140a, always including the PHR 101a. A preferred communication link protocol can be two-way TLS, also known as TLS with client certificate authentication. A preferred configuration would utilize 2048-bit SSL certificates along with 256-bit encryption.

In addition to connections to health organization networks 110, 120, 130, and/or 140, the PHIE system 100 can include one or more patient monitors 160 for collection of real-time biometric and medical data from the patient. A patient monitor can be connected to the PHIE system 100 through a direct connection to central service provider 170 by a communication link related to the patient monitor 160a. Alternately, a patient monitor can be connected to the PHIE system 100 through an indirect connection to central service provider 170 by connection to the patient's personal repository 101 through evolving wireless means, including but not limited to Bluetooth™ technology, which in turn is connected to central service provider 170 by a communication link related to the patient's personal repository 101. Depending on applications available on the personal repository 101, the personal repository 101 can act as a gateway to stream the real-time biometric and medical data from the patient to the central service provider 170, or the real-time biometric and medical data from the patient can be stored in a file 160a on the personal repository 101 for later transmission to the central service provider 170.

In conjunction the collection of real-time biometric and/or medical data from the patient, standardized medical data 150 can be accessed for comparison of the patient's real-time biometric and/or medical data to the expected real-time biometric and/or medical data for the patient to identify any possible health-related abnormalities.

In addition to any of the foregoing, the user can also manually enter personal medical information into the PHR 101a. Such personal medical information can include but is not limited to, blood type, medications, allergies, upcoming medical appointments, emergency doctors contact information, authorized physicians, organ donor information, advance directives. or any other relevant information.

Specific embodiments of the PHIE system 100 are disclosed by combination of all or a portion of the descriptions of each element of the system.

### Method Related to Personal Repository

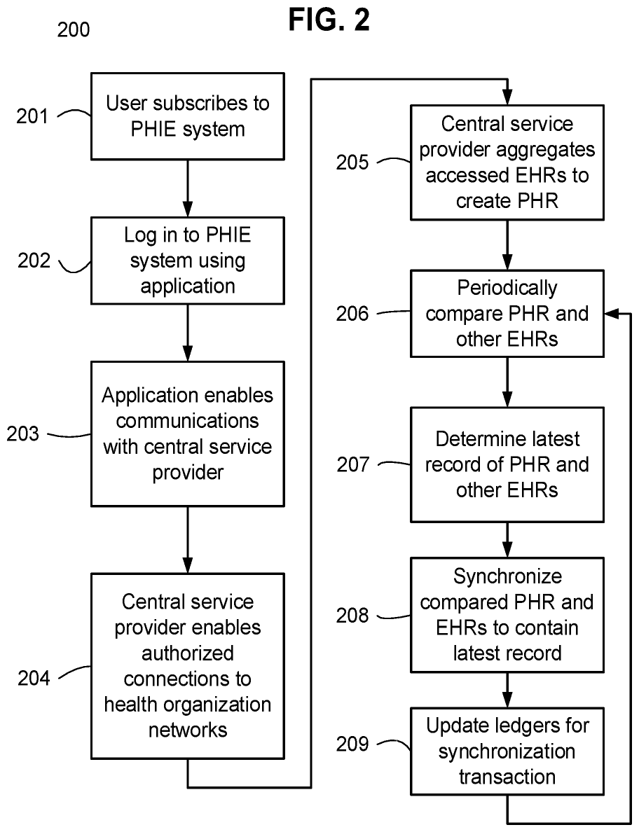

In an embodiment shown in FIG. 2, a method 200 for obtaining a current PHR or personal EHR 101a using the PHIE system 100 is described. A user, or patient, subscribes 201 to the PHIE system 100 through a website associated with the PHIE system or an application associated with the PHIE system. The user subscribes 201 to the to the PHIE system 100 by entering personal data, including but not limited to name, address, gender, blood type, phone number(s), medical insurance information, emergency contact(s), medication(s), allergies, and/or EHR provider(s). In some embodiments, an application installed on a mobile personal repository is preferred.

A user logs 202 into the PHIE system either through a website associated with the PHIE or an application associated with the PHIE. When the user logs 202 into the PHIE, the website associated with the PHIE or an application associated with the PHIE enables 203 a communication link 101b with a central service provider 170. In some embodiments, when a user logs into the PHIE system, the user can elect to maintain the communication link 101b. In other embodiments, the communications link 101b is maintained automatically.

After establishing communication link 101b with the central service provider 170, the central service provider, through one or more APIs hosted by the central service provider, enables 204 one or more communication links 110b, 120b, 130b, and/or 140b with authorized health organization networks 110, 120, 130, and/or 140. Health organization networks are authorized when the user adds such networks during the subscription process. Authorized health organization networks 110, 120, 130, and/or 140 maintain copies of user's EHR 110a, 120a, 130a, and/or 140a. The central service provider 170 aggregates 205 information in the EHRs 110a, 120a, 130a, and/or 140a to create 205 a PHR 101a maintained on the user's personal repository 101.

Once activated and a PHR 101a exists on personal repository 101, the PHIE system 100, periodically and automatically, through APIs hosted on the central service provider 170, compares 206 PHR 101a to one or more of copies of user's EHR 110a, 120a, 130a, and/or 140a. APIs hosted on the central service provider 170 prompt such comparisons automatically at predetermined time intervals. In some embodiments, the time interval can be once every two weeks, once every week, once every 3 days, once every 2 days, once every 24 hours, or once every 12 hours. Any other time interval can conveniently be programmed in order to achieve the desired functionality of PHIE system 100.

Preferably, APIs hosted on the central service provider compares the PHR 101a to all copies of user's EHR 110a, 120a, 130a, and/or 140a available at all health organization networks 110, 120, 130, and/or 140 authorized by the user. Comparison between PHR and other EHRs can be done sequentially or simultaneously. When the PHR 101a and one or more other EHRs 110a, 120a, 130a, and/or 140a are compared, APIs hosted on the central service provider determines 207 which record is later and synchronize 208 the records updating older records such that the PHR 101a and one or more other EHRs 110a, 120a, 130a, and/or 140a all contain the latest information. Ledgers associated with the PHR 101a and one or more other EHRs 110a, 120a, 130a, and/or 140a are then updated 209 to reflect the synchronization transaction.

At times, multiple records may become out of sync, either due to a downtime for part of the system or an update of different records in less time than the time period for the automated synchronization of EHRs. For instance, at a particular time of comparison, PHR 101a contains A, EHR 110a contains A+B, and EHR 120a contains A+C. In this case, EHR 110a and 120a are both later records than PHR 101a. One or more APIs hosted on the central service provider 170 are designed to identify these situations and review the ledgers to identify missing transactions for certain records in order to produce a new record containing A+B+C. Synchronization in this case results in PHR 101a, EHR 110a, and EHR 120a all containing A+B+C. Additionally, data are consolidated and organized into a directory such as, but not limited to, medical history, immunizations, family history, medications, laboratory results, x-rays, and scans, with sources of data identified.

Optionally, the user can also add personal information such as, but not limited to, allergies, medications, organ donor preferences, physical activities, and/or medical appointments, to the PHR 101a via the PHIE application on the user's personal repository 101.

### Method Related to Medical Services

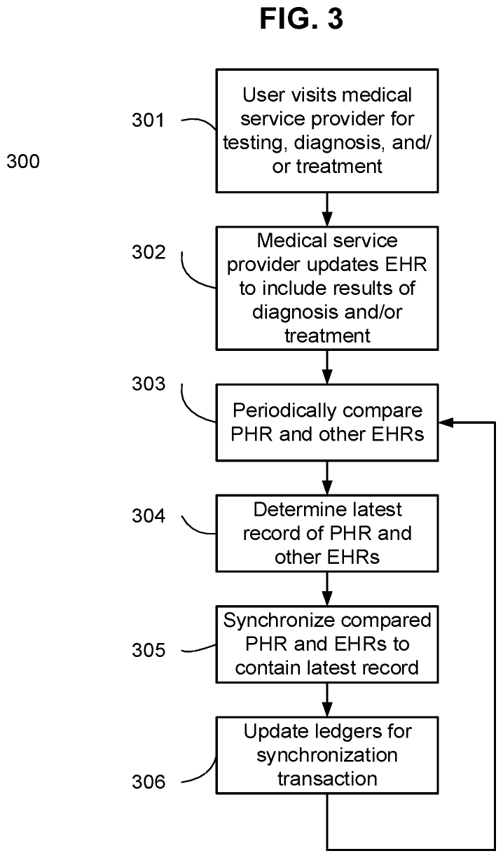

In an embodiment shown in FIG. 3, a method 300 for obtaining a current EHR using the PHIE system 100 is described. A patient can 301 obtain testing, diagnosis, and/or treatment from an medical services provider 111b, which subscribes to a health organization network 110 that maintains an EHR 110a for a patient subscribing to the system. After the medical services provider 110 has completed providing services to the patient, the medical services provider 110 documents the testing, diagnosis, and/or treatment in an appointment record. The medical services provider 110 then adds the appointment record to the patient EHR 110a to create 302 an updated EHR. In some cases, testing, diagnosis, and/or treatment are recorded directly to the EHR to create an updated EHR. Medical services can include ambulatory medical services, routine medical services, or telemedicine services.

The PHIE system 100, periodically and automatically, through APIs hosted on the central service provider 170, compares 303 PHR 101a to one or more of copies of user's EHR 110a, 120a, 130a, and/or 140a and determine 304 the latest EHR version. APIs hosted on the central service provider 170 prompt such comparisons automatically at predetermined time intervals. In some embodiments, the time interval can be once every two weeks, once every week, once every 3 days, once every 2 days, once every 24 hours, or once every 12 hours. Any other time interval can conveniently be programmed in order to achieve the desired functionality of PHIE system 100.

In some embodiments, updating of the patient EHR automatically establishes a communication link 110b between the health organization network 110 and a central service provider 170. An API hosted on the central service provider 170 provides a private subnetwork between the health organization network 110 and at least a patient repository device 101 by establishing a communication link 101b between the central service provider 170 and the patient repository device 101. One or more APIs hosted on the central service provider 170 synchronize 305 the updated EHR and PHR, such that the PHR contains the same information as the updated EHR, and then update 306 ledgers associated with the EHR and the PHR to record the synchronization transaction.

The central service provider 170 can host one or more APIs that provide for inclusion of at least one additional health organization network 120, which maintains a patient EHR 120a. through a communication link 120b. An API hosted on the central service provider 170 provides a private subnetwork between the health organization network 110, the patient repository 101, and the at least one additional health organization network 120 by enabling communication link 120b between the central service provider 170 and at least one additional health organization network 120. One or more APIs hosted on the central service provider 170 synchronize the updated EHR 110a, PHR 101, and the EHR 120a, such that the PHR and EHR 120a contain the same information as the updated EHR, and then update ledgers associated with the EHR 110a, the PHR 101, and EHR 120a to record the synchronization transaction.

A health organization network 110 subscribing to the PHIE system 100, possesses a copy of the patient's EHR 110a. The patient's EHR 110a will match the PHR 101a on the patient's personal repository 110 at the beginning of the visit to a provider of medical services subscribing to health organization network 110. After provision of medical services 110 to the patient, the service provider will document all testing, diagnosis, and/or treatment provided during the visit and create an appointment record, which is later added to the patient EHR 110a, or the patient EHR 110a is updated directly to include such testing, diagnosis, and/or treatment. An application programming interface hosted by the central service provider 170 automatically and periodically checks all service providers subscribing to the PHIE system 100. When the change to patient EHR 110a is detected, the application programming interface will determine the latest EHR version and prompt connections to all other service providers subscribing to the PHIE system 100 through their related communication links to synchronize all other copies of the patient EHR, including the EHR 101a on patient's personal repository 101, to match the updated version of patient electronic record 110a retained by the provider of medical services 110.

In preferred embodiments, the telemedicine platform not only allows the patient and medical services provider the capability of viewing each other during consultations but also allows bidirectional exchange of files, including, but not limited to, x-rays, ultrasound and/or MRI imaging, uploading of biometric readings from wearable biometric patient monitors, and a private messaging capability offline and online. The transcripts of consultations are automatically pushed to the electronic health record system and to the user's personal repository that becomes part of the user's PHR.

### Method Related to Emergency Services

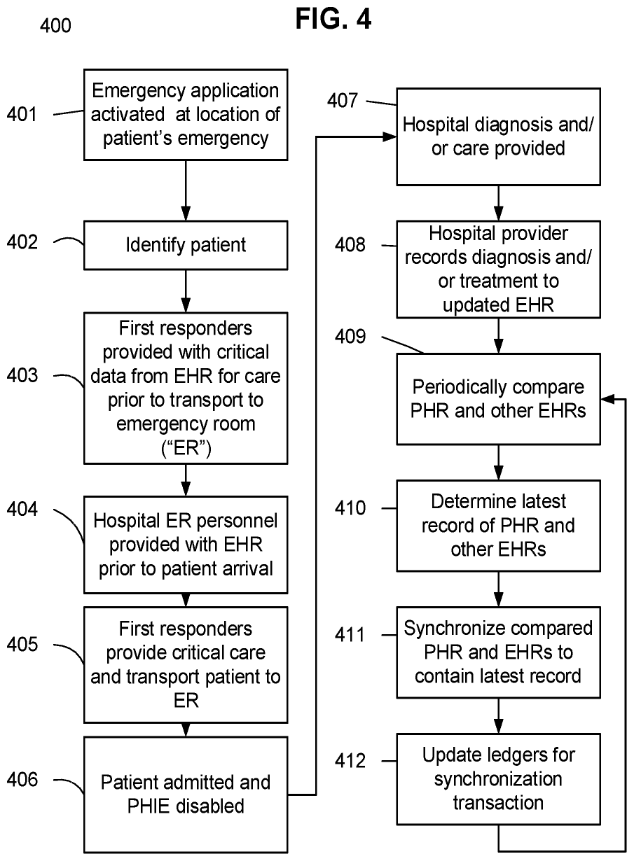

In an embodiment shown in FIG. 4, a method 400 for obtaining a current EHR using the PHIE system 100 is described. When a patient requires emergency medical services, an emergency medical services application on the patient's personal repository 101 can be activated 401 by or on behalf of the patient to facilitate delivery of emergency services to patient. The emergency services can be emergency rooms (at a hospital or other equivalent type facilities) 141, 142 and/or first responders 143. Efficacy of the emergency health information exchange network (EHIE) 140 improves as the membership of hospitals and first responders to the EHIE increases.

The emergency services application automatically establishes a communication link 101c between the patient repository device 101 and an emergency health information exchange network (EHIE) 140 that maintains an EHR 140a for the patient subscribing to the service. If the patient is unconscious, the emergency services application can be activated 401 on the patient repository device 101 by a first responder without the need for an access code or exposing the patient's personal information residing on the patient repository device 101. If the patient personal repository 101 is unavailable or damaged, the emergency services application can be activated 401 on a repository device of the first responders through communication link 143b. In some embodiments, first responders' access to limited and possibly life-saving EHR information might be authorized by city, state, and/or federal authorities as being in the public interest. Identification of an unconscious patient can be further added by an identification bracelet, photo identification, pendant, wallet card, or other similar means to indicate emergency services providers 142, 143 participating in the EHIE system 140 in which a patient is a participant.

A patient must be identified 402. A conscious patient can self-identify. If the patient in unconscious or otherwise unable to self-identify, the EHIE 140 can comprise a communication link to an identification repository. For instance, the identification repository can comprise a master patient index and/or driver registry. The link can be configured to enable the transmissions of user identifying information such as, but not limited to, a photograph of victim, fingerprint, biometric data, and/or other data uniquely identifying an individual. Once identified, all or a portion of the user's EHR, as pre-authorized by the user, can be obtained through communication link 101c to patient personal repository, through communication link 143b to first responders 143, and through communication link 142b to emergency room (ER) 142 (assuming ER 142 is the only ER involved in this example).

Once the emergency services providers are in contact with the ERIE and the patient has been identified, one or more APIs, hosted on the central service provider 170, notifies one or more emergency contacts by email and/or short message system, provides 403 critical patient information to first responders and/or provides a private subnetwork between the patient repository device 101 and the hospital emergency room by establishing communication between the central service provider 170 and the emergency room 142 through a communication link 142b to provide 404 access to EHR 140a. First responders provide 405 critical care and transport patient to emergency room 142. The subnetwork is disconnected 406 upon admission of the patient to the hospital. Hospital personnel provide 407 diagnosis and/or care to patient. Upon completion of emergency services, including related hospital stay, and discharge of patient from the hospital 142, updating the patient EHR 140a is controlled by the emergency services provider 142 to create 408 an updated EHR 140a including all emergency services provided.

The PHIE system 100, periodically and automatically, through APIs hosted on the central service provider 170, compares 409 PHR 101a to one or more of copies of user's EHR 110a, 120a, 130a, and/or 140a and determine 410 the latest EHR version. APIs hosted on the central service provider 170 prompt such comparisons automatically at predetermined time intervals. In some embodiments, the time interval can be once every two weeks, once every week, once every 3 days, once every 2 days, once every 24 hours, or once every 12 hours. Any other time interval can conveniently be programmed in order to achieve the desired functionality of PHIE system 100.

In some embodiments, updating of the patient EHR automatically establishes a communication link 140b between the health organization network 140 and a central service provider 170. An API hosted on the central service provider 170 provides a private subnetwork between the health organization network 140 and at least a patient repository device 101 by establishing a communication link 101b between the central service provider 170 and the patient repository device 101. One or more APIs hosted on the central service provider 411 synchronize the updated EHR and PHR, such that the PHR contains the same information as the updated EHR, and then update 412 ledgers associated with the EHR and the PHR to record the synchronization transaction.

The central service provider 170 can host one or more APIs that provide for inclusion of at least one additional health organization network 120, which maintains a patient EHR 120a. through a communication link 120b. An API hosted on the central service provider 170 provides a private subnetwork between the health organization network 140, the patient repository 101, and the at least one additional health organization network 120 by enabling communication link 120b between the central service provider 170 and at least one additional health organization network 120. One or more APIs hosted on the central service provider 411 synchronize the updated EHR 140a, PHR 101, and the EHR 120a, such that the PHR and EHR 120a contain the same information as the updated EHR, and then update ledgers associated with the EHR 140a, the PHR 101, and EHR 120a to record the synchronization transaction.

A health organization network 140 subscribing to the PHIE system 100, possesses a copy of the patient's EHR 140a. The patient's EHR 140a will match the PHR 101a on the patient's personal repository 140 at the beginning of the visit to a provider of medical services subscribing to health organization network 140. After provision of medical services 140 to the patient, the service provider will document all testing, diagnosis, and/or treatment provided during the visit and create an appointment record, which is later added to the patient EHR 140a, or the patient EHR 140a is updated directly to include such testing, diagnosis, and/or treatment. An application programming interface hosted by the central service provider 170 automatically and periodically checks all service providers subscribing to the PHIE system 100. When the change to patient EHR 140a is detected, the application programming interface will prompt connections to all other service providers subscribing to the PHIE system 100 through their related communication links to synchronize all other copies of the patient EHR, including the EHR 101a on patient's personal repository 101, to match the updated version of patient electronic record 140a retained by the EHIE 140.

### Method Related to Patient Monitors

In another embodiment, one or more patient monitors 160 are used to collect 501 real-time biometric and medical data from a patient. The one or more patient monitors 160 are connected 502 directly 160b or indirectly 160c-101b to a central service provider 170. The real-time biometric and medical data from a patient is transmitted 503 to the central service provider 170 via the enabled connection. One or more APIs hosted on the central service provider 170 perform 504 statistical analysis of patient real-time biometric and medical data and format the results to produce processed patient data. In some embodiments, the processed patient data is added 507 directly to the PHR 101.

In other embodiments. patient's PHR 101a, an application programming interface, hosted on the central service provider 170 provides a connection 150b between the central service provider and a repository of standardized health data 150. Benchmark data is downloaded 505 from the repository of standardized health data 150 to the central service provider 170. One or more APIs on the central service provider 170 compares 506 processed patient data to benchmark data to identify possible health abnormalities in the patient and results are added 507 to the PHR 101a.

While the illustrative embodiments disclosed herein have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside herein, including all features which would be treated as equivalents thereof by those skilled in the art to which the disclosure pertains.

In the preceding description, the present disclosure is described with reference to specifically exemplary embodiments thereof. It will, however, be evident that various modifications and changes may be made thereto without departing from the broader spirit and scope of the present disclosure, as set forth in the claims. The specification and drawings are, accordingly, to be regarded as illustrative and not as restrictive. It is understood that the present disclosure is capable of using various other combinations and embodiments and is capable of any changes or modifications within the scope of the inventive concept as expressed herein.

What is claimed is:

1. A system enabling transmission of a medical history, comprising:
a plurality of health organization networks, at least one of the health organization networks comprising a repository holding a medical history;
a plurality of ledgers encoding changes to the medical history;
a central service provider connected to each of the plurality of health organization networks via a communication link associated with each health organization network; and
a server enhancement hosted by the central service provider, the server enhancement configured to:
provide a private subnetwork between at least a portion of the health organization networks through all or a portion of the communication links; and
direct maintenance of at least one ledger by the health organization networks to reflect changes to the medical history.

2. The system of claim 1, wherein the health organization networks comprise at least one of clinics, hospitals, and offices.

3. The system of claim 1, wherein the communication link comprises one or more of telephone lines, satellite links, and radio transmissions.

4. The system of claim 1, further comprising a network of patient monitors configured to communicate with one or more of the health organization networks via the central service provider through the communication link.

5. The system of claim 1, further comprising a personal repository connection with one or more of the health organization networks via the communication link related to the personal repository and the communication links related to the one or more health organization networks connected through the central service provider.

6. The system of claim 1, further comprising emergency health information exchange network.

7. The system of claim 1, wherein the server enhancement is further configured to transiently store at least one of information identifying a patient, information identifying a document, and a transmitted medical history to facilitate the update of the at least one ledger.

8. A method for obtaining a current electronic health record (EHR), the method comprising:
providing a personal health information exchange (PHIE) to a patient, the PHIE comprising:
a personal repository containing a PHR of the patient;
one or more health organization networks, wherein each network maintains an EHR of the patient and comprises connections to one or more medical services providers selected from one or more of each ambulatory services, emergency services, telemedicine services, routine medical services and/or any combination thereof;
a server enhancement, hosted on a central service provider:
providing a subnetwork between the personal repository and the one or more health organization networks through communication links associated with the personal repository and the one or more health organization networks;
providing automatic review of the subnetwork at a specific time interval to identify a change to any EHR;
providing synchronization of the EHRs in response to new data being added to a single EHR; and
providing for updates to ledgers associated with each EHR to record changes to each EHR;
the patient causing an update to one of the EHRs by obtaining medical services form a medical services provider; and
the patient obtaining and updated PHR after a specific time interval.

9. A method for obtaining a current electronic health record (EHR), the method comprising:
an application on a personal repository automatically enabling a communication link with a central service provider, wherein the personal repository contains a first EHR of a patient; and
a server enhancement, hosted on the central service provider:
providing a private subnetwork between the personal repository and at least a first health organization network by enabling a communication link between the central service provider and the first health organization network, wherein the first health organization network contains a second EHR of a patient;
comparing the first EHR and the second EHR to determine which EHR has the more current data;
synchronizing the first EHR and the second EHR, such that both EHRs contain the more current data; and
updating a ledger associated with each EHR to record the transaction of synchronizing the first EHR and the second EHR.

10. The method of claim 9, wherein the server enhancement, hosted on the central service provider, further:
provides a private subnetwork between the patient repository device, the first health organization network, and at least a second health organization network by enabling a communication link between the central service provider and the second health organization network, wherein the second health organization network contains a third EHR of a patient;
comparing the first EHR, the second EHR, and the third EHR to determine which EHR has the most current data;
synchronizing the first EHR, the second EHR, and the third EHR, such that all three EHRs contain the most current data; and
updating a ledger associated with each EHR to record the transaction of synchronizing all three EHRs.

11. The method of claim 9, wherein the server enhancement, hosted on the central service provider, further:
provides a private subnetwork between the patient repository device, the first health organization network, the second health organization network, and at least a third health organization network by enabling a communication link between the central service provider and the third health organization network, wherein the third health organization network contains a fourth EHR of a patient;
comparing the first EHR, the second EHR, the third EHR, and the fourth EHR to determine which EHR has the most current data;
synchronizing the first EHR, the second EHR, the third EHR, and the fourth EHR, such that all four EHRs contain the most current data; and
updating a ledger associated with each EHR to record the transaction of synchronizing all four EHRs.

12. A method for obtaining a current electronic health record (EHR), the method comprising:
a user creating an updated first EHR on a personal repository by adding information to the first EHR through an application on the personal repository;
the application on the personal repository automatically establishing a communication link with a central service provider; and
a server enhancement, hosted on the central service provider:
providing a private subnetwork between the personal repository and at least a first health organization network by enabling a communication link between the central service provider and the first health organization network, wherein the first health organization network contains a second EHR of a patient;
synchronizing the first EHR and the second EHR, such that both EHRs contain the information of the updated first EHR; and
updating a ledger associated with each EHR to record the transaction of synchronizing the first EHR and the second EHR.

13. A method for obtaining a current electronic health record (EHR), the method comprising:
a patient receiving testing, diagnosis, and/or treatment from a medical services provider, wherein the medical services provider subscribes to a first health organization network, which maintains a first EHR for the patient;
the medical services provider documenting the testing, diagnosis, and/or treatment in the patient EHR to create an updated first EHR;
a server enhancement, hosted on the central service provider:
providing a private subnetwork between the first health organization network and a patient repository device by enabling a communication link between the central service provider and the patient repository device, wherein a PHR resides on the patient repository device;
comparing the updated first EHR and the PHR to identify that the updated first EHR has more current data;
synchronizing the updated first EHR and the PHR, such that both EHRs contain the more current data; and
updating a ledger associated with each EHR to record the transaction of synchronizing the first EHR and the PHR.

14. The method of claim 13, wherein the server enhancement, hosted on the central service provider, further:
provides a private subnetwork between the first health organization network, the patient repository device, and at least a second health organization network by enabling a communication between the central service provider and the second health organization network, wherein the second health organization network maintains a second EHR;
comparing the updated first EHR, the PHR, and the second EHR to identify that the updated first EHR has more current data;
synchronizing the updated first EHR, PHR, and second EHR, such that the PHR and the second EHR each contain the information of the updated first EHR; and
updating a ledger associated with each EHR and the PHR to record the transaction of synchronizing the EHR and the PHR.

15. The method of claim 13, wherein the medical services provider is an ambulatory medical services provider, a telemedicine services provider, or a routine medical services provider.

16. A method for obtaining a current electronic health record (EHR), the method comprising:
providing a personal health information exchange (PHIE) system comprising:
an emergency medical services application, an emergency health information network (EHIE), a patient EHR stored on the EHIE, and a personal repository;
activating the emergency services application;
identifying patient; and
a server enhancement, hosted on a central service provider:
providing first responders with critical data from EHR by enabling a private communication link between the first responders and the ERIE; and
providing emergency room personnel access to EHR by enabling a private communication link between the emergency room personnel and the ERIE;
first responders providing critical care and transporting patient to emergency room;
a second server enhancement, hosted on the central service provider:
disconnecting the PHIE when patient is admitted to emergency room and/or hospital;
emergency room and/or hospital personnel providing diagnosis and/or care to patient;
emergency room and/or hospital personnel recording all emergency medical services provided to patient to create updated EHR; and
a third server enhancement, hosted on the central service provider:
providing a private subnetwork between the EHIE and a patient repository device by enabling a communication link between the central service provider and the patient repository device, wherein a PHR resides on the patient repository device;
comparing the updated EHR and the PHR to identify that the updated EHR has more current data;
synchronizing the updated EHR and the PHR, such that both EHRs contain the more current data; and
updating a ledger associated with the EHR and PHR to record the transaction of synchronizing the EHR and the PHR.

17. The method of claim 16, wherein the first server enhancement, hosted on the central service provider, further provides for:
notifying one or more emergency contacts by email and/or short message system.

18. A method for obtaining a current electronic health record (EHR), the method comprising:
providing a personal health information exchange (PHIE) system comprising a personal health record stored on a personal repository and at least on patient monitor;
a patient monitor collecting real-time biometric and/or medical data from a patient;
delivering real-time biometric and/or medical data from a patient to a central service provider; and
a server enhancement, hosted on the central service provider:
providing for processing, statistical analysis, and formatting of patient real-time biometric and medical data to produce processed patient data; and
providing for addition of the processed patient data to the PHR.

19. The method of claim 18, the server enhancement, hosted on the central service provider, further provides for:
providing a private subnetwork between the patient repository device and a repository of standardized health data by enabling a communication link between the central service provider and a repository of standardized health data;
extracting benchmark data from the repository of standardized health data;
comparing the processed patient data to the benchmark data to identify possible health-related abnormalities with the patient to produce benchmark report; and
providing for addition of the benchmark report to the PHR.
